(19) United States
(12) Patent Application Publication
(10) Pub. No.: US 2005/0163650 A1
(43) Pub. Date: Jul. 28, 2005

(54) Particulate neutralization system for air handling equipment

(76) Inventors: Keith Crawford, Chalfont, PA (US); Timothy Mitchell, Trooper, PA (US)

Correspondence Address: Michael G. Crilly, Esq., 104 South York Road, Hatboro, PA, 19040, US

(21) Appl. No.: 10/762,919

(22) Filed: Jan. 22, 2004

(51) Int. Cl. A61L 9/00

(52) U.S. Cl. 422/4; 422/24; 422/120; 422/121

(57) ABSTRACT

The present invention is a neutralization system for particulates including germs, organisms, and airborne pathogens. The invention includes a duct, a lamp with at least one ultraviolet tube therein, an optically transmissible element, and a light panel. The duct has an exterior surface with openings and an interior volume through which an air stream is directed. The lamp is fastened to the exterior surface of the duct over a first opening. The optically transmissible element is secured between lamp and duct so as to prevent the air stream from contacting the ultraviolet tubes within the lamp. The light panel is comprised of a frame about a porous mat composed of a plurality of end emitting optical fibers. The panel is slidably disposed through a second opening so as to bisect the air stream. A first end of each end emitting optical fiber is positioned so as to allow ultraviolet light from the lamp to enter the fiber. Ultraviolet light is projected from a second end of each fiber within the porous mat so that individual light beams overlap to form a contiguous field. In alternate embodiments, ultraviolet light is communicated into a single light panel from two or more lamps. In yet other embodiments, two or more light panels are provided within a single duct. The present invention is applicable to a variety of ducts, examples including but not limited to cooling, heating and ventilation, through which a contaminated air stream is directed.

### CROSS REFERENCE TO RELATED APPLICATIONS

None.

### FEDERALLY SPONSORED RESEARCH OR DEVELOPMENT

None.

### BACKGROUND OF THE INVENTION

1. Field of the Invention

The present invention generally relates to a system for the neutralization of particulates including germs, organisms, and airborne pathogens. Specifically, the invention includes a plurality of optical fibers communicating light from one or more remotely disposed ultraviolet tubes into an air stream, wherein the ultraviolet light is emitted from one end of each fiber to provide a plurality of beams to form a field of ultraviolet radiation through which the air stream passes.

2. Description of the Related Art

While ultraviolet lamps are recognized for their germicidal properties, effective implementations of such devices within air handling equipment for the neutralization of organic particulates have been limited due to a well known practical limitation. Namely, the intensity of light emitted from an ultraviolet lamp, and thereby the effectiveness of such devices, decreases dramatically with distance.

The related arts include a variety of filtration, neutralization and disinfection devices to improve the coupling of ultraviolet light onto organic particulates within an air stream. The most common approach is to reduce the distance between light source and particulates by placing one or more ultraviolet tubes within the air stream in an air duct, as described by Vilarasau Alegre (U.S. Pat. No. 6,653,647), Guzorek (U.S. Pat. No. 6,630,678), Fend et al. (U.S. Pat. No. 6,627,000), Brumett (U.S. Pat. No. 6,619,063), Palestro et al. (U.S. Pat. No. 6,497,840), Fencl et al. (U.S. Pat. No. 6,372,186), Bach (U.S. Pat. No. 5,894,130), Fencl et al. (U.S. Pat. No. 5,866,076), Meinzer et al. (U.S. Pat. No. 5,865,959), Summers (U.S. Pat. No. 5,837,207), Berman et al. (U.S. Pat. No. 5,766,455), Von Glehn (U.S. Pat. No. 5,681,374), Morrow et al. (U.S. Pat. No. 5,656,242), Mazzilli (U.S. Pat. No. 5,523,057), Pick et al. (U.S. Pat. No. 5,330,722), Gazzano (U.S. Pat. No. 5,112,370), and Horng (U.S. Pat. No. 4,931,654).

The related arts also include non-germicidal inventions having an ultraviolet lamp within an air stream to remove inorganic compounds. For example, Fleck et al. in U.S. Pat. No. 5,564,065 teaches a carbon monoxide air filter comprised of an ultraviolet lamp surrounded by a matrix of fibrous material, typically fiberglass, holding a photo-excitable powder thereon. Ultraviolet light is communicated to the photo-excitable powder via a side-glow fiber embedded within the fibrous material so as to excite the powder which oxidizes carbon monoxide to form carbon dioxide.

The above referenced related arts are plagued by technical problems that reduce the effectiveness and limit the life span of ultraviolet tubes.

It is well known that ultraviolet tubes generate an electrostatic field attracting particulates which accumulate and form a coating thereon. This coating over time impedes ultraviolet emissions and frustrates the neutralization of airborne particles.

It is likewise known that the performance of ultraviolet tubes is temperature sensitive. A moving air stream reduces tube temperature thereby decreasing the effective wavelength of ultraviolet emissions which reduces the efficient neutralization of airborne particles. Furthermore, a lower operating temperature shortens tube life. Additional tubes are typically introduced to offset reductions in tube performance by increasing the intensity of ultraviolet light within the air stream. However, this approach increases operating and maintenance costs.

It is also known that ultraviolet tubes are susceptible to mechanical failure when impacted by debris within an air stream. Furthermore, such tubes are degraded and damaged by moisture and other contaminates within an air stream.

What is currently required is a particulate neutralization system capable of communicating ultraviolet light from a remote source into an air stream so as to avoid the problems found in the related arts.

What is also required is a particulate neutralization system capable of emitting ultraviolet light within a duct through which an air stream must pass.

### SUMMARY OF THE INVENTION

An object of the present invention is to provide a particulate neutralization system wherein ultraviolet light from an external source is communicated into a duct via a plurality of fiber optic cables.

A further object of the present invention is to provide a particulate neutralization system wherein a field of ultraviolet radiation is achieved via a plurality of individual beams of light.

A further object of the present invention is to provide a field of ultraviolet radiation so as to neutralize particulates within an air stream.

In preferred embodiments, the invention includes a duct, a lamp having at least one ultraviolet tube therein, an optically transmissible element, and a light panel. The duct has an exterior surface with two openings and an interior volume through which an air stream is directed. The lamp is fastened to the exterior surface of the duct over a first opening. The optically transmissible element is secured between lamp and duct so as to prevent the air stream from contacting the ultraviolet tubes within the lamp. The light panel has a porous mat composed of a plurality of end emitting optical fibers and a frame about its perimeter. The panel is slidably disposed through a second opening and removably secured within the duct so as to bisect the air stream. A first end of each end emitting optical fiber is positioned so as to allow ultraviolet light from the lamp to enter the fiber. Ultraviolet light is projected from a second end of the same fiber within the porous mat so that individual light beams form a field within the duct.

In alternate embodiments, the invention includes a duct, two or more lamps each having at least one ultraviolet light, two or more optically transmissible elements, and a single light panel. Each lamp is fixed to the exterior surface of the duct over an opening with one optically transmissible element secured there between. The light panel is composed of a frame and a porous mat of end emitting optical fibers. The panel is slidably disposed into the duct and removably secured within the duct so as to bisect the air stream. Lamps are arranged about the light panel so as to communicate ultraviolet light into the optical fibers. Ultraviolet light is emitted from the optical fibers so as to provide a plurality of ultraviolet beams within the porous mat forming a field within the duct through which the air stream passes.

In other embodiments, the invention includes a duct, two or more lamps each having at least one ultraviolet light, two or more optically transmissible elements, and two or more light panels. Each lamp is fixed to the exterior surface of the duct over an opening with one optically transmissible element secured there between. Each light panel is composed of a frame and a porous mat of end emitting optical fibers. Panels are separately disposed within and slidably disposed into the duct and removably secured within the duct so as to bisect the air stream. At least one lamp communicates ultraviolet light into the optical fibers comprising each porous mat. Ultraviolet light is emitted from the optical fibers so as to provide a plurality of ultraviolet beams within each porous mat to form one or more fields within the duct through which the air stream passes.

A variety of optional arrangements are possible for the above described embodiments. For example, the optically transmissible element may be a lens to either focus or spread light from a lamp prior to entering the optical fibers. It is likewise possible to have a lens at one or both ends of each optical fiber within the porous mat to focus or disperse light. Furthermore, filter elements may be positioned upstream, downstream and/or between light panels to remove particulates prior to and/or after neutralization.

Several advantages are offered by the present invention. The invention allows ultraviolet light to be communicated into a duct from a remote source while avoiding the loses inherent to remote placement. The invention avoids both cooling and environmental conditions that limit tube life. The light panel is both durable and washable.

### BRIEF DESCRIPTION OF THE DRAWINGS

The invention will now be described in more detail, by way of example only, with reference to the accompanying drawings, in which:

FIG. 1 is a cross-sectional view of a particulate neutralization system showing a light panel within a duct and a lamp with an ultraviolet tube attached to the exterior of the duct at one end of the light panel.

FIG. 2 is a cross-sectional view at line 2-2 of the particulate neutralization system of FIG. 1.

FIG. 3 is an enlarged cross-sectional view of lamp and light panel showing ultraviolet light communicated through an optically transmissible element into a plurality of optical fiber.

FIG. 4 is a schematic representation of several optical fibers from a porous mat of a light panel whereby each launches an ultraviolet beam thereafter forming a contiguous field.

FIG. 5a is an elevation view of an optical fiber from a porous mat showing a conical-shaped envelope of ultraviolet light at the fiber end.

FIG. 5b is an elevation view of the optical fiber of FIG. 5a having a lens increasing the angle of the conical-shaped envelope of ultraviolet light at the fiber end.

FIG. 5c is an elevation view of the optical fiber of FIG. 5a having a lens decreasing the angle of the conical-shaped envelope of ultraviolet light at the fiber end.

FIG. 6 is an alternate embodiment of the particulate neutralization system shown in FIG. 2 having two parallel disposed lamps illuminating optical fibers within a light panel.

FIG. 7 is an alternate embodiment of the particulate neutralization system shown in FIG. 2 having two perpendicularly disposed lamps illuminating optical fibers within a light panel.

FIG. 8 is an alternate embodiment of the particulate neutralization system shown in FIG. 2 having three lamps illuminating optical fibers within a light panel.

FIG. 9 is an alternate embodiment of the particulate neutralization system shown in FIG. 1 having an optional pre-filter and an optional post-filter.

FIG. 10 is an alternate embodiment of the particulate neutralization system shown in FIG. 1 having two light panels within a duct with particulate filters upstream, downstream and between the panels.

### REFERENCE NUMERALS

1 Particulate neutralization system
2 Ultraviolet tube
3 Bracket
4 Optically transmissible element
5 Duct
6 Lamp
7 Air stream
8 Interior volume
9 Exterior surface
10 Light panel
11 First opening
12 Frame
13 Porous mat
14 Optical fiber
15 Light
16 First end
17 Second end
18 Ultraviolet beam
19 Contiguous field
20 Lens
21 Target distance, d
22 Spot diameter, D
23 Angle, α
24 Flange
25 Second opening
26 Upstream
27 Downstream
28 Pre-filter
29 Post-filter
30 Intermediate filter
31 Width
33 Height
34 Gasket
35 Flange
36 Fiber end

### DESCRIPTION OF THE INVENTION

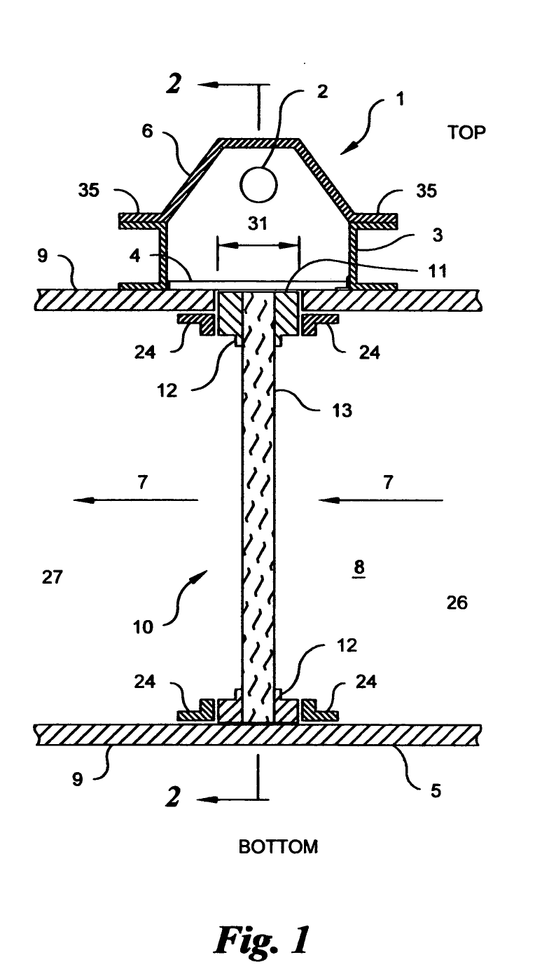

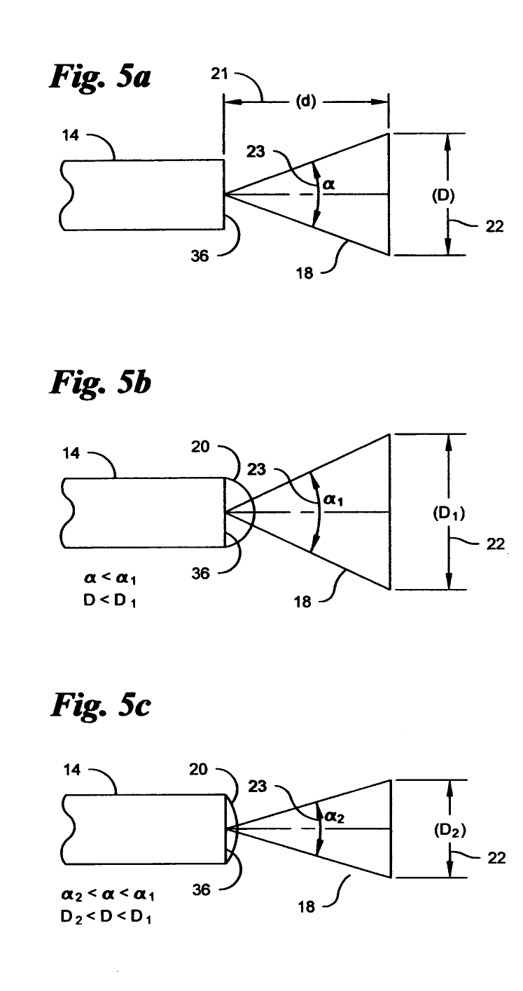

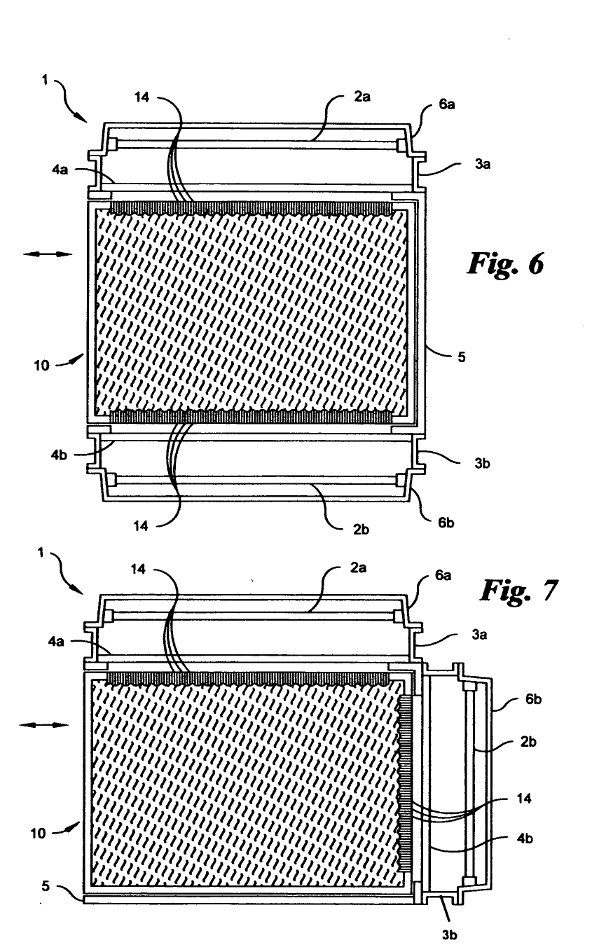

FIGS. 1-3 describe one embodiment of the particulate neutralization system 1 comprising a duct 5, a lamp 6 having at least one ultraviolet tube 2, an optically transmissible element 4, and a light panel 10. FIG. 5 graphically describes alternate embodiments wherein a lens 20 is attached to each optical fiber 14 within the light panel 10. FIGS. 6-10 describe several alternate embodiments having multiple lamps 6, multiple light panels 10, and additional filtration elements.

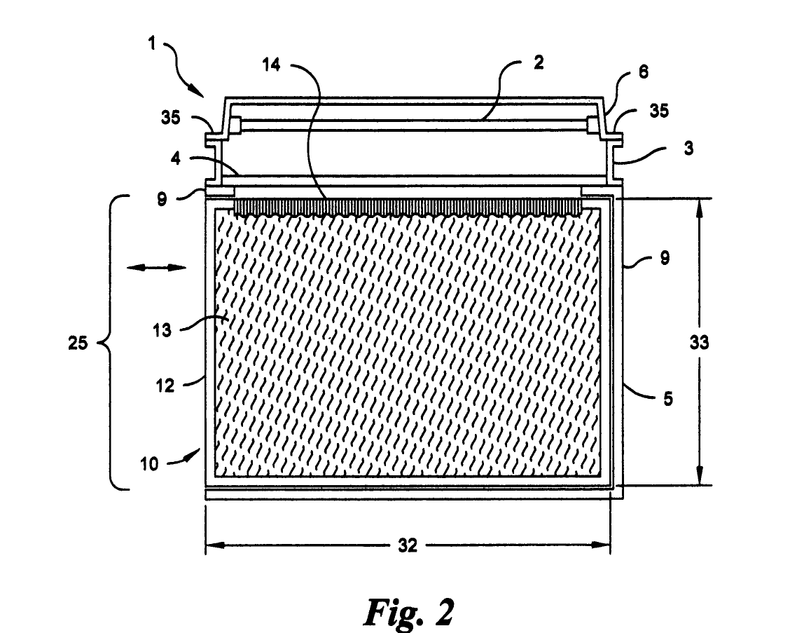

Referring now to FIGS. 1-2, a duct 5 is shown having a light panel 10 within the interior volume 8 of the duct 5 in a perpendicular arrangement so as to bisect the air stream 7 into upstream 26 and downstream 27 components. The duct 5, as is understood in the art, is a conduit through which the air stream 7 is directed. The duct 5 has a rectangular-shaped first opening 11 and a rectangular-shaped second opening 25 both aligned in a lengthwise fashion perpendicular to the flow direction of the air stream 7, as shown in FIGS. 1 and 2, respectively. A lamp 6 is secured to the exterior surface 9 of the duct 5 and aligned lengthwise with the first opening 11. It is preferred to have the first opening 11 nearly as long as the length 32 and as wide as the width 31 of the light panel 10. The light panel 10 is inserted into the duct 5 through the second opening 25. It is also preferred to have the second opening 25 slightly larger than the height 33 and slightly wider than the width 31 of the light panel 10. Clearance between light panel 10 and second opening 25 should allow sliding motion there between yet minimize leakage of ultraviolet light and air stream 7 from the interior volume 8. It is likewise possible to have a removal panel with seal covering the second opening 25 to prevent leakage of ultraviolet light and air stream 7.

Referring to FIG. 1, two pairs of right-angle flanges 24 are either tack welded or mechanically fastened to the duct 5 top and bottom within the interior volume 8 so as to provide a guide way for the placement of the light panel 10 and prevent movement thereof within the duct 5 due to loading by the air stream 7.

The lamp 6 houses one or more ultraviolet tubes 2 which emit ultraviolet light in a directed fashion. A flange 35 about the perimeter of the lamp 6 is fastened to a four-sided bracket 3, having a c-shaped cross section as shown in FIGS. 1-2, via screws or comparable fasteners. Thereafter, the bracket 3 is fastened to the exterior surface 9 of the duct 5 so as to completely surround the first opening 11. The described arrangement between lamp 6, bracket 3, and first opening 11 insures the directed projection of ultraviolet light from the lamp 6 onto the light panel 10 adjacent to the first opening 11. A continuous bead of silicon-based caulk may be desired along contacting surfaces between flange 35 and bracket 3 and bracket 3 and duct 5.

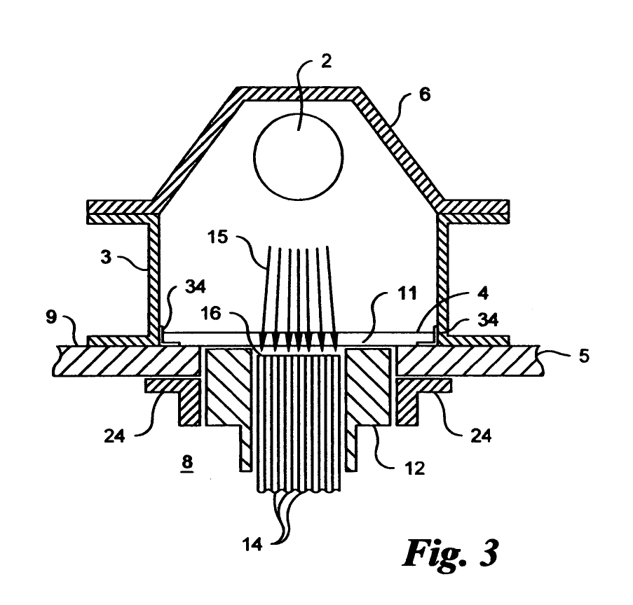

The optically transmissible element 4 is secured to the exterior surface 9 of the duct 5 bounded by the bracket 3, also shown in FIGS. 1-2. Referring to FIG. 3, a gasket 34, preferably an ultraviolet resistant adhesive, is applied about the perimeter of the optically transmissible element 4 so as to contact and bond with both duct 5 and bracket 3 thereby sealing the duct 5 and preventing the air stream 7 from exiting the duct 5 through the first opening 11. While a variety of commercially available transparent, ultraviolet transmissible glasses and plastics were found to be adequate for the optically transmissible element 4, preferred embodiments included a planar-shaped float glass.

The number of ultraviolet tubes 2 within a lamp 6 and their operational characteristics, namely voltage, power and wavelength, are dependent on the cross sectional dimensions of the duct 5, flow rate through the duct 5, and quantity and type of particulates within the air stream 7. As such, a variety of commercially available ultraviolet tubes 2 and lamps 6 are applicable to the present invention. A high output utility fixture having two germicidal bulbs, model no. UHFO26-2-120, sold by the American Ultraviolet Company located in Lebanon, Ind. is a non-limiting example.

Referring again to FIG. 2, the light panel 10 includes a rectangular-shaped frame 12 disposed about the perimeter of a porous mat 13. The frame 12 has an inwardly disposed u-shape cross section along sides not immediately adjacent to a lamp 6, as shown in FIG. 1. The frame 12 is composed of a rigid or semi-rigid material, non-limiting examples including polyethylene, polypropylene and water-resistant cardboard. The porous mat 13 is secured to the frame 12 within the u-shaped structure via an adhesive or mechanical fasteners.

The porous mat 13 is composed of a plurality of end emitting optical fibers 14 oriented in a weave-like fashion or randomly intertwined in a mesh like-fashion. Gaps or spaces between optical fibers 14 within the weaver or mesh allow the air stream 7 to traverse the light panel 10 while minimizing the pressure drop between upstream 26 and downstream 27. In yet other embodiments, it was desired to minimize the gaps or spaces between optical fibers 14 within the porous mat 13 so as to also trap particulates therein.

Optical fibers 14 direct ultraviolet light from a first end 16 to a second end 17 without exit there between. Such end emitting optical fibers 14 are required to communicate ultraviolet light for an extended period without degradation and to be sufficiently flexible to resist breakage during fabrication of the porous mat 13. A single-mode fiber having a high numerical aperture and composed of a hard clad silica sold by the 3M Company with model number FT400-URT is an exemplary optical fiber 14.

Referring now to FIG. 3, the first end 16 of each optical fiber 14 is arranged in a parallel fashion and thereafter bundled along a side of and passing through the frame 12. Optical fibers 14 were adhesively bonded to one another and to the frame 12. Optical fibers 14 are oriented towards the ultraviolet tube 2 so that light 15 is communicated into the first end 16 of each optical fiber 14.

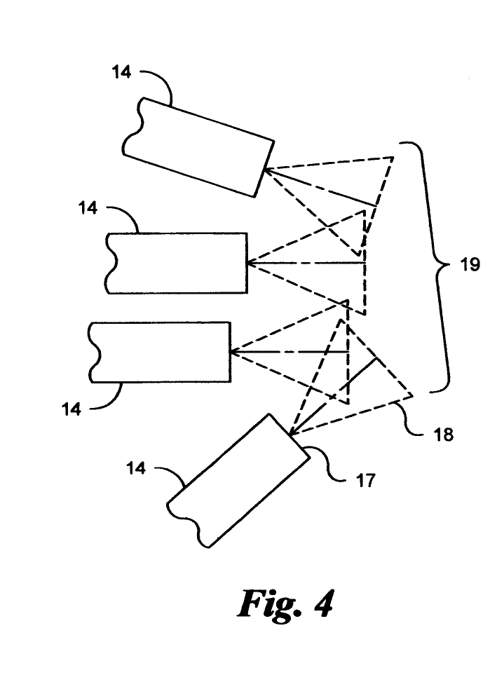

Referring now to FIG. 4, the second end 17 of each optical fiber 14 resides within the porous mat 13 so as to allow a conical-shaped ultraviolet beam 18 to be launched therefrom. Optical fibers 14 may be oriented so that ultraviolet beams 18 are launched in an ordered or random pattern. It is desired that ultraviolet beams 18 overlap to form a contiguous field 19 within and/or adjacent to the porous mat 13, as graphically represented in FIG. 4. The contiguous field 19 is positioned within the interior volume 8 of the duct 5 so as to bisect the air stream 7 thereby insuring ultraviolet light is communicated to particulates therein.

The efficient coupling of light 15 into the first end 16 of the optical fiber 14 is improved or tailored via the optically transmissible element 4. For example, the planar-disposed optically transmissible element 4 may be shaped to function as a lens so as to focus or to diffuse light 15 from the ultraviolet tube 2 before it enters the first end 16 of the optical fiber 14.

The efficient coupling of light 15 into and ultraviolet beam 18 out of the optical fiber 14 may be tailored via a lens 20. Referring now to FIGS. 5b-5c, a lens 20 is shown at the fiber end 36 of an exemplary optical fiber 14. In FIG. 5b, the lens 20 diffuses the ultraviolet beam 18 launched from the optical fiber 14 so as to have a larger angle 18 and spot diameter 22 at a target distance 21 than the optical fiber 14 without a lens 20 in FIG. 5a. It is likewise possible for the lens 20 described in FIG. 5b to increase light 15 communicated into the optical fiber 14 by the lamp 6. In FIG. 5c, the lens 20 focuses the ultraviolet beam 18 so as to have a smaller angle 18 and spot diameter 22 at a target distance 21 than the optical fiber 14 without lens 20 in FIG. 5a. It is possible to lense the fiber end 36 via chemical mill and mechanical grinding techniques.

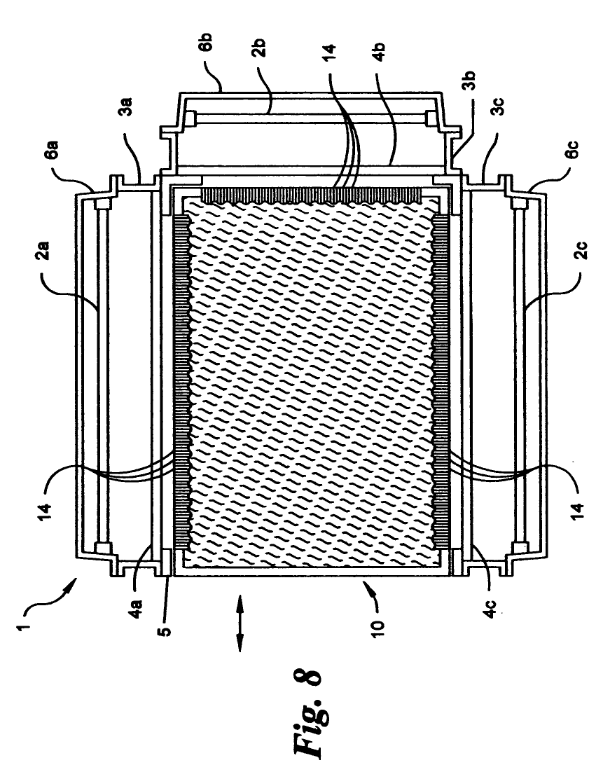

In some applications, it may be desired to communicate light 15 into the optical fibers 14 of the porous mat 13 via two or more ultraviolet lamps 6a-6c. FIGS. 6-7 show embodiments having two lamps 6a, 6b attached to the duct 5 via brackets 3a, 3b, as described above. In FIG. 6, lamps 6a, 6b are arranged parallel at opposite sides of the light panel 10 thereby communicating light 15 into the optical fibers 14. In FIG. 7, lamps 6a, 6b, are positioned in a perpendicular arrangement about the light panel 10 thereby communicating light 15 into the optical fibers 14. Each lamp 6a, 6b includes one or more ultraviolet tubes 2 radiating light 15 at one or more wavelengths. FIG. 8 shows an embodiment having three lamps 6a-6c, aligned with three sides of a light panel 10 and communicating light 15 into the optical fibers 14. Ultraviolet tubes 2 are shielded from the air stream 7 via an optically transmissible element 4a, 4b, and 4c. Light 15 is communicated into optical fibers 14 as described above for FIG. 3.

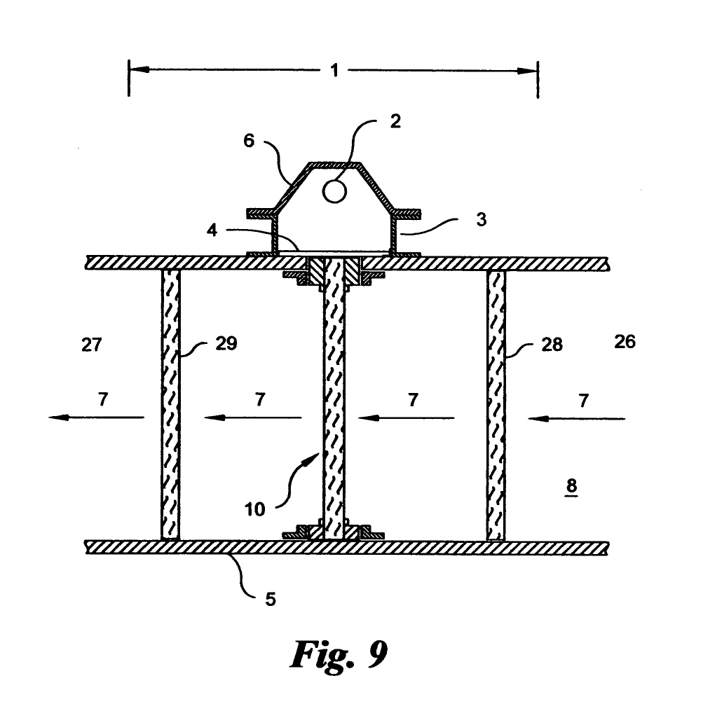

In some embodiments, it may be desired to remove particulates within the air stream 7 prior to and/or after the light panel 10. Referring now to FIG. 9, the particulate neutralization system 1 includes a single light panel 10 within a duct 5 having an optional pre-filter 28 secured within the same duct 5 via techniques understood in the art and upstream 26 from the light panel 10. Likewise, an optional post-filter 29 is shown secured within the same duct 5 via techniques understood in the art and downstream 27 from the light panel 10. A variety of particulate filtration elements are applicable to the pre-filters 28 and post-filters 29, including but not limited to commercially available pleated and HEPA filters.

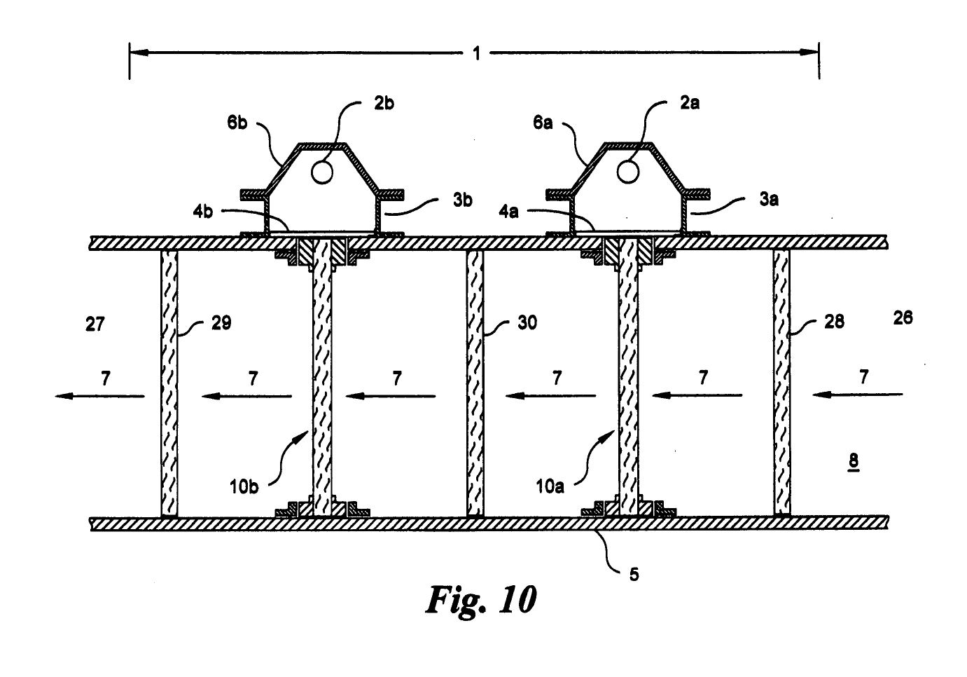

In alternate embodiments, it may be desired to include two or more light panels 10 disposed in a parallel fashion along a single duct 5. Referring now to FIG. 10, a particulate neutralization system 1 is shown having two light panels 10a, 10b disposed along a duct 5 with an optional pre-filter 28 upstream 26, an optional post-filter 29 downstream 27 and an optional intermediate filter 30 between the light panels 10a, 10b. Each light panel 10a, 10b communicates light 15 from at least one ultraviolet lamp 6, as described above for FIGS. 2, 6, 7, and 8, into the interior volume 8 thereby forming one or more contiguous fields 19 across the cross section of the duct 5. For example, ultraviolet beams 18 from two closely spaced light panels 10a, 10b might combine to form a single contiguous field 19. It is likewise possible that two or more light panels 10 are sufficiently separated so that each provides a contiguous field 19.

The description above indicates that a great degree of flexibility is offered in terms of the present invention. Although the present invention has been described in considerable detail with reference to certain preferred versions thereof, other versions are possible. Therefore, the spirit and scope of the appended claims should not be limited to the description of the preferred versions contained herein.

1. A particulate neutralization system comprising:
(a) a duct having an exterior surface with a first opening and a second opening and an interior volume through which an air stream is directed;
(b) a lamp having at least one ultraviolet tube therein, said lamp fixed to said exterior surface over said first opening;
(c) an optically transmissible element secured to said duct between said lamp and said interior volume so as to prevent said air stream from contacting said ultraviolet tube; and
(d) a light panel comprising a frame and a porous mat attached to said frame, said light panel slidably disposed through said second opening, removably secured to said duct and bisecting said air stream, said porous mat composed of a plurality of end emitting optical fibers, a first end of each said end emitting optical fiber disposed towards said lamp with said optically transmissible element there between, a second end of each said end emitting optical fiber disposed within said porous mat so as to communicate a plurality of ultraviolet beams to form a field through which said air stream passes.

2. The particulate neutralization system of claim 1, wherein said optically transmissible element is a lens.

3. The particulate neutralization system of claim 1, further comprising a pre-filter upstream from said light panel.

4. The particulate neutralization system of claim 1, further comprising a post-filter downstream from said light panel.

5. The particulate neutralization system of claim 1, wherein said first end of said end emitting optical fibers has a lens.

6. The particulate neutralization system of claim 1, wherein said second end of said end emitting optical fibers has a lens.

7. A particulate neutralization system comprising:
(a) a duct having an exterior surface and an interior volume through which an air stream is directed;
(b) at least two lamps each having at least one ultraviolet tube therein, said lamps fixed to said exterior surface with each over an opening in said duct;
(c) at least two optically transmissible elements wherein one said optically transmissible element is secured to said duct between each said lamp and said interior volume so as to prevent said air stream from contacting said ultraviolet tubes; and
(d) a light panel comprising a frame and a porous mat attached to said frame, said light panel slidably disposed into said interior volume, removably secured to said duct and bisecting said air stream, said porous mat composed of a plurality of end emitting optical fibers, a first end of each said end emitting optical fiber disposed towards one said lamp with one said optically transmissible element there between, a second end of each said end emitting optical fiber disposed within said porous mat so as to communicate a plurality of ultraviolet beams to form a contiguous field through which said air stream passes.

8. The particulate neutralization system of claim 7, wherein said optically transmissible element is a lens.

9. The particulate neutralization system of claim 7, further comprising a pre-filter upstream from said light panel.

10. The particulate neutralization system of claim 7, further comprising a post-filter downstream from said light panel.

11. The particulate neutralization system of claim 7, wherein said first end of said end emitting optical fibers has a lens.

12. The particulate neutralization system of claim 7, wherein said second end of said end emitting optical fibers has a lens.

13. A particulate neutralization system comprising:
(a) a duct having an exterior surface and an interior volume through which an air stream is directed;
(b) at least two lamps each having at least one ultraviolet tube therein, said lamps fixed to said exterior surface with each over an opening in said duct;
(c) at least two optically transmissible elements wherein one is secured to said duct between each said lamp and said interior volume so as to prevent said air stream from contacting said ultraviolet tubes; and
(d) at least two light panels each comprising a frame and a porous mat attached to said frame, said light panels slidably disposed into said interior volume, removably secured to said duct and bisecting said air stream, each said porous mat composed of a plurality of end emitting optical fibers, a first end of each said end emitting optical fiber disposed towards at least one said lamp with one said optically transmissible element there between, a second end of each said end emitting optical fiber disposed within one said porous mat so as to communicate a plurality of ultraviolet beams to form at least one field through which said air stream passes.

14. The particulate neutralization system of claim 13, wherein at least one said optically transmissible element is a lens.

15. The particulate neutralization system of claim 13, further comprising a pre-filter upstream from said light panels.

16. The particulate neutralization system of claim 13, further comprising a post-filter downstream from said light panels.

17. The particulate neutralization system of claim 13, further comprising an intermediate-filter disposed between two said light panels.

18. The particulate neutralization system of claim 13, wherein said first end of said end emitting optical fibers has a lens.

19. The particulate neutralization system of claim 13, wherein said second end of said end emitting optical fibers has a lens.

20. A particulate neutralization method comprising the steps of
(a) communicating a plurality of ultraviolet light beams into an air stream;
(b) coupling said ultraviolet light beams into a field;
(c) passing said air stream through said field; and
(d) exposing a plurality of particulates within said air stream to said field.

21. A light panel for neutralizing particulates within an air stream comprising:
(a) a frame; and
(b) a porous mat composed of a plurality of end emitting optical fibers attached to said frame, a first end of each said end emitting optical fiber disposed within said frame so as to receive light from a light source, a second end of each said end emitting optical fiber disposed within said porous mat so as to communicate a plurality of ultraviolet beams to form a field.
